(19) United States
(12) Patent Application Publication
(10) Pub. No.: US 2017/0251949 A1
(43) Pub. Date: Sep. 7, 2017

(54) SYNCHRONIZING AN MR IMAGING PROCESS WITH ATTAINMENT OF THE BREATH-HOLD STATE

(71) Applicants: Flavio Carinci, Erlangen (DE); Wilhelm Horger, Schwaig (DE); Mario Zeller, Erlangen (DE)

(72) Inventors: Flavio Carinci, Erlangen (DE); Wilhelm Horger, Schwaig (DE); Mario Zeller, Erlangen (DE)

(21) Appl. No.: 15/447,834

(22) Filed: Mar. 2, 2017

(30) Foreign Application Priority Data
Mar. 2, 2016 (DE) 102016203416.2

(51) Int. Cl. A61B 5/055; A61B 5/00; A61B 5/11

(52) U.S. Cl. CPC A61B 5/055; A61B 5/11; A61B 5/6802

(57) ABSTRACT

A method for synchronizing an MR imaging process with a breathing rest state of a patient during an examination using held breath is provided. In the method, an instruction is output to the patient to hold his breath. In addition, the respiratory behavior of the patient is identified in real time. An MR imaging process is started according to the identified respiratory behavior. A breathing synchronization device and a magnetic resonance imaging system are also provided.

This application claims the benefit of DE 10 2016 203 416.2, filed on Mar. 2, 2016, which is hereby incorporated by reference in its entirety.

### BACKGROUND

The present embodiments relate to synchronizing a magnetic resonance (MR) imaging process with a breathing rest state during an examination using held breath.

In many examinations using magnetic resonance imaging (e.g., in the chest and abdominal region), the respiratory movement of the patient produces motion artifacts. One approach to reducing these artifacts is to perform the MR imaging process during a time interval in which the patient is holding his breath. For example, before an image acquisition sequence is started, acoustic instructions are given automatically to the patient to hold his breath during exhalation or inhalation. The actual MR imaging process (e.g., the scan process) is performed immediately thereafter. Patients do not always follow the instructions given as intended, however. In fact patients need additional time until they are completely still. The first image acquisitions in a series of image acquisitions are thus usually impaired by the movement of the patient. This phenomenon is a problem especially for volumetric interpolated breath-hold examination (VIBE), because in this imaging process, a reference image for the parallel image reconstruction is typically recorded in the first seconds. If the reference image data is impaired by the movement of the patient, this impacts on the quality of the reconstruction of the complete, subsequent image acquisition. The impairment of the image quality may be ascribed to mis-encoding of the MR resonance signals associated with a specific region of the body. If the patient moves, the position of the body region concerned changes, with the result that this region is exposed to a different gradient strength that when in a rest position. This causes a change in the Larmor frequency in the region that has moved and ultimately to an incorrect spatial association of the detected signals in the image reconstruction.

This presents the problem of defining an MR imaging method in which image artifacts are reduced in image acquisitions in the chest region and abdominal region.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for detecting a breathing rest state during an examination using held breath is provided.

In the method according to one or more of the present embodiments for synchronizing an MR imaging process with a breathing rest state of a patient during an examination using held breath, the patient is given an instruction (e.g., automatically) to hold his breath. As an alternative to an automated output of the instruction to the patient, operating personnel may actuate a switch to trigger a breath-hold instruction. The patient typically needs a certain personal reaction time before actually holding his breath after receiving the breath-hold instruction. In order to avoid starting an imaging procedure too early (e.g., in a phase in which the patient continues to breathe and his body is making a respiratory movement), according to one or more of the present embodiment, the respiratory behavior of the patient is now identified in real time. In other words, the respiratory behavior of the patient is identified as part of the imaging process in time-correlation with (e.g., close in time to) the time at which the breath-hold instruction is output and/or in a preset time span (e.g., a fixed time window). The time window may cover a time period of 1.5 s, for example. In general terms, the timing of the time window may be designed to be close enough to the event of the breath-hold instruction to leave, after the identification, enough time still for the MR imaging process during the phase of the breathing rest state.

The MR imaging process is started (e.g., automatically) according to the identified respiratory behavior. In other words, the start time for the MR imaging process is set such that the acquisition process (e.g., the excitation and read-out of RF signals as part of the imaging process) takes place during a breathing rest state. The breathing rest state has been initiated by the breath-hold instruction, which may have been issued automatically. The breathing rest state (e.g., the breath-hold state) may be the condition of a patient in which the patient is currently not moving between two respiratory movements and is holding his breath. Alternatively, the breathing rest state may be the condition of the patient in which the patient is not inhaling after exhaling, but is remaining still so that, for example, there is very little or no movement of the ribcage. The method according to one or more of the present embodiments achieves individualized adjustment of the start time of an MR imaging process, allowing a sharp reduction in the frequency with which image artifacts arise as a result of a respiratory movement of the patient. In addition, monitoring of the respiratory movement may be confined to a time period after or around the time at which a breath-hold instruction is output. The combination of temporary monitoring of the breathing of a patient and the output of a breathing instruction allows a longer MR image acquisition time compared with continuous monitoring of the respiratory movement of the patient without a breath-hold instruction, because after a breath-hold instruction, the patient makes an effort to hold his breath as long as possible. Thus, the rest period is extended considerably compared with continuous respiratory activity. Thus, the MR imaging process may be performed in longer time periods and need not be interrupted as often as in a procedure in which respiratory activity is continuous. This reduces the total time of the examination process, because there is a better ratio of imaging periods to breaks.

The breathing synchronization device according to one or more of the present embodiments includes an instruction output unit for, for example, the automatic output of an instruction to the patient to hold his breath. The instruction output unit may be electrically connected, for example, to a loudspeaker situated in the region of the magnetic resonance scanner of a magnetic resonance imaging machine so that a patient inside the magnetic resonance scanner may be given instructions acoustically. The breathing instructions or breathing commands are communicated to the patient located inside the magnetic resonance scanner by an audio communications unit. In addition, the breathing synchronization device according to one or more of the present embodiments includes a respiratory-movement identification unit for identifying the respiratory behavior of the patient in real time. A start synchronization unit for starting the MR imaging process according to the identified respiratory behavior is also part of the breathing synchronization device according to one or more of the present embodiments.

The magnetic resonance imaging system according to one or more of the present embodiments includes an RF transmit system, a gradient system, and a controller configured to control the RF transmit system and the gradient system for the purpose of performing a desired measurement based on a specified pulse sequence. In addition, the magnetic resonance imaging system includes the breathing synchronization device according to one or more of the present embodiments. The breathing synchronization device may be part of the controller of the magnetic resonance imaging system according to one or more of the present embodiments, for example. It is also possible for just some of the device to be part of the controller and for the device to be distributed over a plurality of units. The breathing synchronization device may also be fitted as an add-on unit or retrofit kit to a magnetic resonance imaging system or be integrated therein.

Many of the components of the breathing synchronization device according to one or more of the present embodiments may be embodied in the form of software components. This applies, for example, to parts of the instruction output unit, of the respiratory-movement identification unit, and of the start synchronization unit. However, some of these components may also be implemented in the form of software-aided hardware (e.g., FPGAs or the like) when especially fast calculations are to be provided. Likewise, the required interfaces may be configured as software interfaces (e.g., if all that is involved is a transfer of data from other software components). The interfaces may also be configured, however, as hardware-built interfaces driven by suitable software.

An implementation largely in software has the advantage that even magnetic resonance imaging systems or controllers of magnetic resonance imaging systems that are already in use may be easily upgraded by a software update to work in the manner according to one or more of the present embodiments. In this respect, a corresponding computer program product including a non-transitory computer-readable storage medium storing a computer program that may be loaded directly into a memory device of a magnetic resonance imaging system or into a memory device of a controller of a magnetic resonance imaging system according to one or more of the present embodiments is provided. The computer program includes program segments having instructions to perform all the acts of the method according when the computer program is executed in the controller and/or in the magnetic resonance imaging system.

Such a computer program product may include, in addition to the computer program, if applicable, additional elements such as, for example, documentation and/or additional components, including hardware components such as, for example, hardware keys (e.g., dongles) for using the software.

For the purpose of transfer to the memory device of the controller of the magnetic resonance imaging system and/or to the memory device of the magnetic resonance imaging system, a machine-readable medium (e.g., a non-transitory computer-readable storage medium such as a memory stick, a hard disk, or any other portable or permanently installed data storage medium) may be used. Program segments of the computer program are stored on the non-transitory computer-readable storage medium. The program segments may be downloaded and executed by a processing unit of the controller and/or of the magnetic resonance imaging system. For this purpose, the processing unit may include, for example, one or more interacting microprocessors or the like.

The claims in one category of claims may also be developed in a similar way to the dependent claims in another category of claims. Within the scope of the invention, the various features of different exemplary embodiments and claims may also be combined to create new exemplary embodiments.

In one embodiment of the method for synchronizing an MR imaging process with a breathing rest state of a patient during an examination using held breath, during identification of the respiratory behavior, a respiratory curve is initially recorded at least after the output of the instruction to the patient. The respiratory curve may also be recorded already in advance (e.g., shortly before the output of the instruction to the patient) or during or after the time at which the instruction is output to the patient. The first two variants have the additional advantage that the respiratory cycle of the patient before the time of the intended breath-hold period of the patient may be used as reference data and/or calibration data for the subsequent detection act. The recorded respiratory curve is used as the basis for identifying a time at which a breathing rest state of the patient starts. In addition, the described method may also be preceded by a form of training or learning process in which the reaction response of the patient and the respiratory curve values that typify different phases of the respiratory cycle are identified. These values may then be used as reference values or calibration values in the real-time identification of the start time of the breath-hold.

In one embodiment of the method for synchronizing an MR imaging process with a breathing rest state of a patient during an examination performed using held breath, the MR imaging process is started based on the time identified as the start of the breath-hold state (e.g., immediately after the identified time). Optimum use is thereby made of the time period of the breath-hold state of the patient for the MR imaging process.

In one variant of the method for synchronizing an MR imaging process with a breathing rest state of a patient during an examination using held breath, in the situation in which a start time for a breath-hold state of the patient has not been identified within a preset time interval (e.g., 1.5 s), the MR imaging process is started automatically. Using this variant, for the situation in which the patient does not actually hold his breath, the image quality is not reduced compared to a conventional procedure. If, however, the patient has followed the breath-hold instruction and the breath-hold was merely not detected, then an improved image quality may still be enjoyed because of the breath-hold state.

As an alternative for the situation mentioned, the MR imaging process is interrupted and the operating personnel are automatically given notification of the interruption. Then, the operating personnel may confer with the patient and resolve any problems or misunderstandings that may have arisen. As another alternative, the method is repeated, and in addition, a reminder is automatically communicated to the patient to follow carefully the instruction to the patient. Thus, the behavior of the patient may hence be influenced during the imaging process, thereby improving the image quality.

In a variant of the method for synchronizing an MR imaging process with a breathing rest state of a patient during an examination using held breath, identifying the respiratory behavior of the patient in real time includes monitoring the respiratory cycle of the patient using an external apparatus.

The external apparatus may include, for example, a breathing belt or a sensor (e.g., based on an electromagnetic reflection or a radar technology). An external apparatus of this type may be used to record the respiratory movement of the patient independently of the processes of the magnetic resonance imaging system. This provides that the monitoring process need not be integrated directly (e.g., as a pulse sequence) in the operating procedure of the magnetic resonance imaging system.

Identifying the respiratory behavior of the patient in real time may also include monitoring the respiratory cycle of the patient using an internal mechanism. For example, this may include outputting an MR navigator sequence (e.g., including a series of gradient echo sub-sequences without phase encoding). For the output of an MR navigator sequence, a suitable MR navigator sequence may be stored in the controller or breathing synchronization device being used, or may be downloaded thereto before the start of the actual MR imaging process. In this variant, advantageously no hardware additional to the magnetic resonance imaging system already used for the MR imaging process is needed for monitoring the respiratory cycle.

Alternatively or additionally, the navigator may be positioned on the diaphragm, and image-based tracking of the movement of the diaphragm may be performed. This likewise produces a respiratory curve. This procedure is used in the PACE technique, for example.

The respiratory curve may be recorded by acquiring the MR signal at the k-space center at a plurality of time instants and extracting the phase component of the acquired MR signal. For a signal encoded in one spatial direction, the phase in the k-space center equals approximately the phase averaged over all the points in the spatial domain.

The respiratory curve may be acquired by capturing the MR signal using a body coil, applying a Fourier transform, and calculating a signal-weighted sum of the signal phases for each readout time interval. It is thereby possible to acquire a smoother respiratory curve, which may be used to give an even more precise prediction of the start time for the phase in which the patient is in the breath-hold state.

The Fourier transform is used to transform the k-space raw data acquired during acquisition of the respiratory curve into the spatial domain. The phase may be identified in a more stable manner in the spatial domain. As already explained, the phase in the k-space center equals the average of the phases of all the points in the spatial domain. Weighted averaging may be performed if the identification is performed in the spatial domain. For example, points outside the body (e.g., associated with high noise) may be excluded, and weighting may be performed based on signal intensity, for example.

In a specific embodiment of the method, identifying the start time of a breathing rest state of the patient includes at least one of the following method acts: comparing time-dependent standard deviation values of the phase values of the acquired MR signals, which have been obtained using a sliding time window; comparing time-derivative values of the phase values of the acquired MR signals with derivative values from earlier in time; comparing time-derivative values of the phase values of the acquired MR signals with a threshold value; and comparing the absolute values of the phase values of the acquired MR signals with a reference value.

In this context, a sliding time window may be a time window that moves in time with the recording of the respiratory curve, with the result that the values recorded for each time window are used in the calculation of the values described. The first variant, which relates to comparing time-dependent standard deviation values of the phase values of the acquired MR signals that have been obtained using a sliding time window may also be performed without the prior acquisition of reference values, because the comparison values are obtained directly from the recorded respiratory curve. The same applies to the second variant, in which time-derivative values of the phase values of the acquired MR signals are compared with derivative values from earlier in time. For the third and fourth variants, threshold values or reference values may be obtained, for example, by a learning phase that precedes the imaging process, with the respiratory behavior of the patient concerned being investigated by prior acquisition of a respiratory curve.

### BRIEF DESCRIPTION OF THE DRAWINGS

FIG. 1 shows an exemplary graph illustrating a respiratory curve as a function of a sampling time;

FIG. 2 is a flow diagram illustrating one embodiment of a method for synchronizing an MR imaging process with a breathing rest state of a patient during an examination using held breath;

FIG. 3 contains two graphs that show exemplary respiratory curves recorded and analyzed as part of the method;

FIG. 4 is a block diagram illustrating one embodiment of a breathing synchronization device; and

FIG. 5 shows one embodiment of a magnetic resonance imaging system.

### DETAILED DESCRIPTION

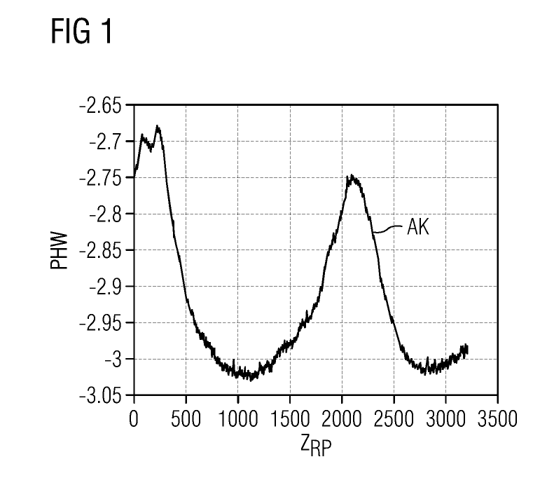

FIG. 1 shows an example of a respiratory curve AK acquired using a magnetic resonance (MR) navigator sequence containing a gradient echo without phase encoding and having a repetition time TR=5 ms and a flip angle of 4°, with 3200 repetitions performed in 16 seconds. In FIG. 1, the phase values PHW of acquired MR resonance signals from the MR navigator sequence are plotted as a function of the current repetition ZRP. The curve was acquired by simple extraction of the phase component of the MR signal, obtained from a body coil, from the k-space center for each individual point in time. A smoother curve may be obtained, for example, by using surface coils and/or using a Fourier transform and by the signal-weighted summation of the image phases for each echo. The respiratory graph presented in FIG. 1 shows even, flat respiration of a patient without held breath.

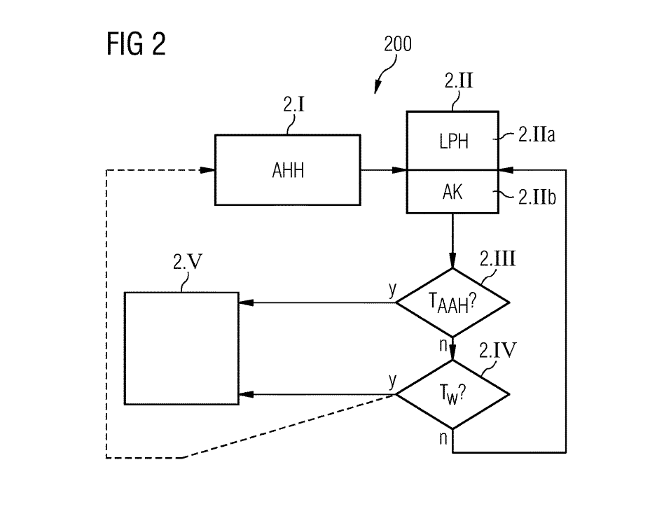

FIG. 2 shows a flow diagram 200 illustrating one embodiment of a method for synchronizing an MR imaging process with a breathing rest state of a patient during an examination using held breath. In act 2.I, first an acoustic instruction AAH is issued automatically to the patient to hold his respiratory movement. In act 2.II (see act 2.IIb), a respiratory curve AK is acquired immediately after the output of the breath-hold instruction AAH. Optionally, in act 2.IIa, the respiratory curve may be recorded for a learning phase LPH. The recording of the respiratory curve AK may be based, for example, on the acquisition of MR signals using an MR navigator sequence. The phase values PHW reconstructed from the MR signals are in this case used as the amplitude values of the respiratory curve AK, which may be associated with the particular phase of the respiratory movement. For example, phase values PHW at which the breathing rest state starts are identified. Alternatively, derivatives of the respiratory curve AK or other values that are correlated with the start of the breath-hold state may also be calculated. Reference values RW are thereby formed, which are used for comparison with the recorded respiratory curve AK during the subsequent real-time curve recording and analysis for identifying a start time of a breath-hold state. In act 2.IIb, the respiratory curve AK is recorded after the end of the learning phase LPH, again on a breath-hold instruction AAH, as given in act 2.I. Alternatively or additionally, in act 2.II, the respiratory curve AK may also be acquired using external units such as a breathing belt, for example, or using sensors (e.g., based on an electromagnetic reflection or a radar technology).

In act 2.III, the recorded respiratory curve AK is analyzed by comparing the recorded phase values PHW of the respiratory curve AK with the reference values RW recorded during the learning phase. Based on the analysis of the recorded respiratory curve AK, the current stage of the respiratory process of the patient may be identified. For example, it is identified in act 2.III whether the patient is starting to hold his breath. This process may be associated with a plateauing of the recorded respiratory curve AK, for example. For the case in which the start time T_{AAH} of the breath-hold state has not yet been reached, which is labeled “n” in FIG. 2, then in act 2.IV, a check is performed as to whether a preset maximum wait time T_{W}, for example 1.5 s, has been reached yet. For the case in which the preset maximum wait time T_{W} has not been reached yet, which is labeled “n” in FIG. 2, then a return is made to act 2.II or 2.IIb, and recording of the respiratory curve AK continues. If, however, in act 2.IV, the preset wait time T_{W} has already been reached, which is labeled “y” in FIG. 2, then a move is made to act 2.V, in which the actual MR image acquisition of a field of view FOV of the patient is started. As an alternative to automatically starting the MR image acquisition after the wait time T_{W}, it is also possible to return to act 2.I, which is indicated in FIG. 2 by a dashed arrow, and a new breath-hold instruction AAH may be output. Then a respiratory curve AK is recorded again in act 2.II or 2.IIb etc. If it was identified in act 2.III that the start time T_{AAH} of the breath-hold state has been reached, which is labeled “y” in FIG. 2, then a move is likewise made to act 2.V, and the actual MR image acquisition of a field of view FOV of the patient is started.

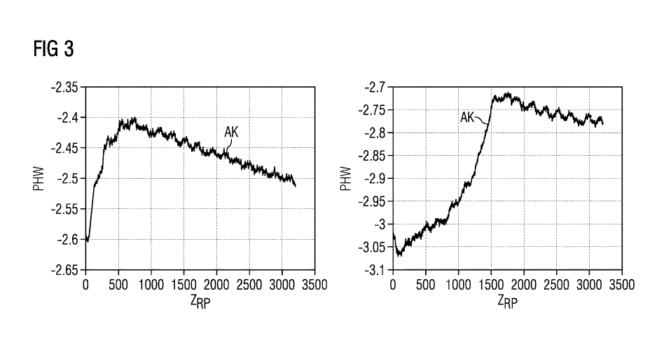

FIG. 3 shows two respiratory curves AK of a patient. For the respiratory curve AK in the left-hand graph, the patient was given a breath-hold instruction AAH immediately before recording of the respiratory curve AK was started. The reaction time T_{AAH} of the patient may be identified from the plateauing of the curve at about 600 repetition cycles, which for a repetition time TR=5 ms gives a reaction time of about 3 s. With regard to the respiratory curve AK in the right-hand graph of FIG. 3, the patient was given a breath-hold instruction AAH approximately after 1000 repetition cycles. As is evident from the right-hand graph, the respiratory curve AK plateaus at around 1500 repetition cycles, and therefore a reaction time T_{AAH} of about 2.5 s may be identified from the graph given a repetition time TR=5 ms. The start time of the actual MR image acquisition may thus be set very precisely using the recorded respiratory curve AK. The time at which the patient holds his breath may be identified in various ways. For example, a standard deviation of phase values PHW that have been recorded in a sliding time window may be calculated. If a value of the standard deviation drops below a preset threshold value, for example, because the respiratory curve AK plateaus as a result of the start of the breath-hold state of the patient, then this is used as the trigger signal for starting the MR imaging process. Alternatively, a time derivative of the recorded phase values PHW may be calculated. If the value for the derivative of the recorded phase values PHW drops below a preset threshold value, then it may be assumed therefrom that the patient is holding his breath and the MR imaging process may be started. Alternatively, instead of using a threshold value, the comparisons mentioned may also be performed with values that were recorded at earlier points in time during the real-time recording of the respiratory curve. If in a learning phase LPH, phase values PHW were associated with specific breathing states, then the phase values PHW may be compared directly with a reference value RW or reference-value interval obtained in the learning phase, and the start time T_{AAH} of the MR imaging process may be set based on the comparison result.

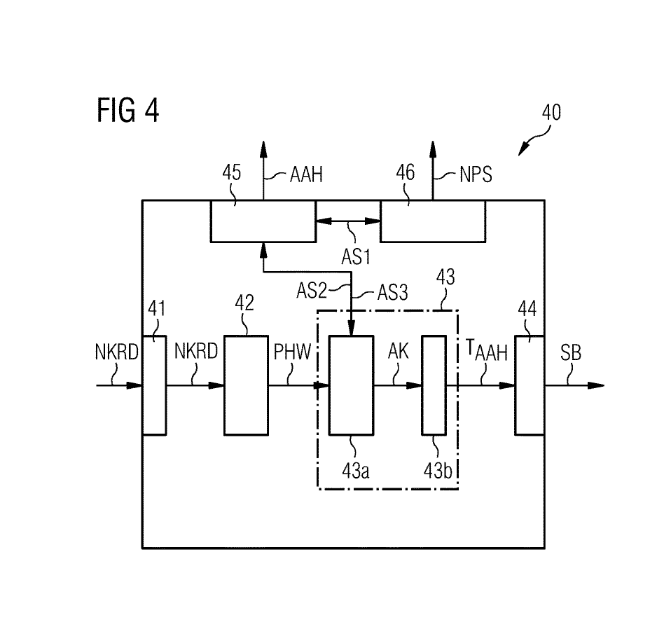

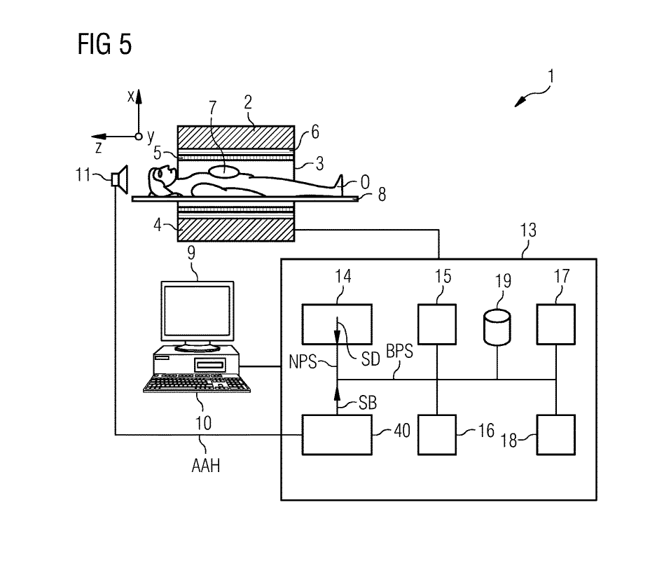

FIG. 4 shows schematically an exemplary breathing synchronization device 40. The breathing synchronization device 40 may be part of a controller (e.g., a processor) of a magnetic resonance imaging system, for example (see FIG. 5). The breathing synchronization device 40 includes a raw-data acquisition unit 41, which receives raw data (e.g., navigator k-space data NKRD) recorded during a navigator image acquisition. The raw data NKRD is transmitted to a phase-value calculation unit 42, which extracts phase values PHW from the raw data NKRD. The phase values PHW are then passed to a respiratory-movement identification unit 43. The respiratory-movement identification unit 43 includes a respiratory-curve recording unit 43a, which is configured to use the received phase values PHW as the basis for recording in real time a respiratory curve AK of a patient under examination. The respiratory-movement identification unit 43 also includes an analysis unit 43b that identifies, based on the recorded respiratory curve AK, the time T_{AAH} at which the patient starts actually to hold his breath. Based on this time information T_{AAH}, a trigger signal SB, for example, may be transmitted via a start synchronization unit 44 to a sequence control unit (not shown), which starts the output of an MR pulse sequence for an image recording of a region of interest of a patient in response to the trigger command. The breathing synchronization device 40 also includes an instruction output unit 45 for the automatic output of an instruction AAH to the patient O to hold his breath. The instruction output unit 45 is connected to the respiratory-movement identification unit 43 in order to trigger the respiratory-movement identification unit 43 to start operating (e.g., by transmitting a trigger signal AS2) after a breath-hold instruction AAH has been issued. The breathing synchronization device 40 also includes a navigator sequence generator 46, which is used to generate a navigator pulse sequence NPS in order to acquire navigator k-space data NKRD from the respiratory movement of the patient. The navigator sequence generator 46 transmits to the instruction output unit 45 a trigger signal AS1 in order to cause the instruction output unit 45 to output automatically a breath-hold instruction AAH (e.g., after the output of the navigator pulse sequence NPS). Other points in time for the output of the breath-hold instruction AAH are also possible. For example, the breath-hold instruction may also be output earlier in time than the output of the navigator pulse sequence NPS. In the event that a preset time has elapsed without the patient holding his breath, the opposite case, in which a trigger signal AS3 is transmitted from the respiratory-movement identification unit 43 to the instruction output unit 45, is also possible in order to output a breath-hold instruction AAH again to the patient.

FIG. 5 shows one embodiment of an MR machine 1. The MR machine 1 includes the actual magnetic resonance scanner 2 containing an examination space 3 or patient tunnel, into which an object under examination O may be moved on a couch 8 (e.g., a patient or subject under examination in whose body the object under examination such as a specific organ is located).

The magnetic resonance scanner 2 is equipped in the usual manner with a main magnetic field system 4, a gradient system 6, and an RF transmit antenna system 5 and an RF receive antenna system 7. In the exemplary embodiment shown, the RF transmit antenna system 5 is a whole-body coil that is fixed in the magnetic resonance scanner 2, whereas the RF receive antenna system 7 consists of local coils (represented in FIG. 5 by a single local coil) arranged on the patient or subject under examination. The whole-body coil may also be used as the RF receive antenna system, and the local coils may be used as the RF transmit antenna system, provided these coils may each be switched into different operating modes.

The MR machine 1 also includes a central controller 13 (e.g., a processor) that is used to control the MR machine 1. This central controller 13 includes a sequence control unit 14 (e.g., a sequence controller) for controlling the pulse sequence. This is used to control the sequence of radio frequency (RF) pulses and gradient pulses according to a selected imaging sequence. Such an imaging sequence may be specified, for example, in a measurement or control protocol. Different control protocols for different measurements are typically stored in a memory 19 and may be selected by an operator (and possibly modified if required), and then used to perform the measurement.

For the output of the individual RF pulses, the central controller 13 includes an RF transmit unit 15, which generates and amplifies the RF pulses and feeds the RF pulses into the RF transmit antenna system 5 via a suitable interface (not shown in detail). The controller 13 includes a gradient system interface 16 for controlling the gradient coils of the gradient system 6. The sequence controller 14 communicates in a suitable manner (e.g., by sending out sequence control data SD) with the RF transmit unit 15 and the gradient system interface 16 for the emission of the pulse sequences. The controller 13 also includes an RF receive unit 17 (likewise communicating with the sequence controller 14 in a suitable manner) for the purpose of coordinated acquisition of magnetic resonance signals received by the RF transmit antenna system 7 (e.g., of raw data). A reconstruction unit 18 receives the acquired image data and reconstructs the MR image data therefrom. This image data may then be stored, for example, in a memory 19 and/or, for the case of navigator image data, processed in a breathing synchronization device 40 according to one or more of the present embodiments in order to start an MR imaging process in the phase of a breathing rest state of the patient O. The breathing synchronization device 40 gives a control command SB, for example, to the sequence controller 14 in order to start sequence control data SD relating to a navigator pulse sequence NPS or an MR image acquisition pulse sequence BPS. In addition, the breathing synchronization device 40 also has a connection to an audio communications unit 11 on the magnetic resonance scanner 2, in order to communicate breath-hold instructions AAH to the patient O.

The central controller 13 may be operated by a terminal having an input unit 10 and a display unit 9, via which an operator may hence also operate the entire MR machine 1. MR images may also be displayed on the display unit 9. The input unit 10, if applicable in combination with the display unit 9, may be used to plan and start measurements, and, for example, to select and, if applicable, modify suitable control protocols containing suitable measurement sequences, as described above.

The MR machine 1 according to one or more of the present embodiments and, in particular, the controller 13 may also include a plurality of further components that are not shown here in detail but are typically present in such machines (e.g., components such as a network interface to connect the entire machine to a network and to be able to transfer raw data, image data, parameter maps, other data such as patient-related data or control protocols, or any combination thereof).

The principles of how suitable raw data may be acquired by applying RF pulses and generating gradient fields, and how MR images may be reconstructed from the raw data, are known to a person skilled in the art and are not explained further here. Likewise, a large variety of measurement sequences such as, for example, EPI measurement sequences or measurement sequences for generating diffusion-weighted images are known in principle to a person skilled in the art.

The method and devices described above are merely exemplary embodiments, and the invention may be modified by a person skilled in the art without departing from the scope of the invention insofar as this is defined by the claims. For instance, the method for synchronizing an MR imaging process with a breathing rest state of a patient during an examination using held breath and the breathing synchronization device 40 have primarily been described with reference to acquisition of a respiratory curve AK using a navigator pulse sequence. The invention is not limited to this use, however; a respiratory curve may also be acquired using external detection such as, for example, breathing belts or sensors. The use of the indefinite article “a” or “an” does not rule out the possibility of there also being more than one of the features concerned. Likewise, the term “unit” does not exclude the possibility that the unit consists of a plurality of components, which may also be spatially distributed if applicable.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for synchronizing an MR imaging process with a breathing rest state of a patient during an examination using held breath, the method comprising:
outputting an instruction to the patient to hold breath;
identifying a respiratory behavior of the patient in real time; and
starting the MR imaging process according to the identified respiratory behavior.

2. The method of claim 1, wherein identifying the respiratory behavior comprises:
recording a respiratory curve at least after the output of the instruction to the patient;
identifying a start time of a breathing rest state of the patient based on the recorded respiratory curve.

3. The method of claim 2, wherein the MR imaging process is started based on the identified start time (T_{AAH}), preferably immediately after the identified time (T_{AAH}).

4. The method of claim 3, wherein the MR imaging process is started immediately after the identified start time.

5. The method of claim 2, wherein when a start time (T_{AAH}) for a breath-hold state of the patient has not been identified within a preset time interval:
the MR imaging process is started automatically; or
the MR imaging process is interrupted, and the operating personnel are automatically given notification of the interruption; or
the method is repeated, and a reminder is automatically communicated to the patient to follow carefully the instruction to the patient.

6. The method of claim 1, wherein identifying the respiratory behavior of the patient in real time comprises monitoring the respiratory cycle of the patient using an external apparatus.

7. The method of claim 6, wherein the external apparatus comprises a breathing belt or a sensor, preferably based on an electromagnetic reflection or a radar technology.

8. The method of claim 7, wherein the sensor is based on an electromagnetic reflection or a radar technology.

9. The method of claim 2, wherein identifying the respiratory behavior of the patient in real time comprises monitoring the respiratory cycle of the patient using an internal mechanism.

10. The method of claim 9, wherein monitoring the respiratory cycle of the patient using the internal mechanism comprises outputting an MR navigator sequence, the MR navigator sequence comprising a series of gradient echo sub-sequences without phase encoding.

11. The method of claim 9, wherein recording the respiratory curve comprises acquiring MR signals at a k-space center at a plurality of time instants and extracting a phase component of the acquired MR signals.

12. The method of claim 11, wherein recording the respiratory curve further comprises capturing MR signals using a body coil, applying a Fourier transform and calculating a signal-weighted sum of signal phases for each readout time interval, or a combination thereof.

13. The method of claim 12, wherein identifying the start time of a breathing rest state of the patient comprises:
comparing time-dependent standard deviation values of the phase values of the acquired MR signals, which have been obtained using a sliding time window;
comparing time-derivative values of the phase values of the acquired MR signals with derivative values from earlier in time;
comparing time-derivative values of the phase values of the acquired MR signals with a threshold value;
comparing absolute values of the phase values of the acquired MR signals with a reference value; or
any combination thereof.

14. A breathing synchronization device comprising:
an instruction output unit configured to output an instruction to a patient to hold breath;
a respiratory-movement identification unit configured to identify a respiratory behavior of the patient in real time; and
a start synchronization unit configured to start an MR imaging process according to the identified respiratory behavior.

15. A magnetic resonance imaging system comprising:
an RF transmit system;
a gradient system; and
a controller configured to:
control the RF transmit system and the gradient system to perform a desired measurement based on a specified pulse sequence; and
a breathing synchronization device comprising:
an instruction output unit configured to output an instruction to a patient to hold breath;
a respiratory-movement identification unit configured to identify a respiratory behavior of the patient in real time; and
a start synchronization unit configured to start an MR imaging process according to the identified respiratory behavior.

16. A computer program product comprising a non-transitory computer-readable storage medium that stores instructions executable by a controller, a magnetic resonance imaging system, or the controller and the magnetic resonance imaging system to synchronize an MR imaging process with a breathing rest state of a patient during an examination using held breath, the instructions comprising:
outputting an instruction to the patient to hold breath;
identifying a respiratory behavior of the patient in real time; and
starting the MR imaging process according to the identified respiratory behavior.

17. In a non-transitory computer-readable storage medium that stores instructions executable by a processor of a controller of a magnetic resonance imaging system to synchronize an MR imaging process with a breathing rest state of a patient during an examination using held breath, the instructions comprising:
outputting an instruction to the patient to hold breath;
identifying a respiratory behavior of the patient in real time; and
starting the MR imaging process according to the identified respiratory behavior.
